(19) United States
(12) Patent Application Publication
(10) Pub. No.: US 2022/0363620 A1
(43) Pub. Date: Nov. 17, 2022

(54) AN ISOMERIZATION PROCESS OF PRODUCT OBTAINED FROM BISPHENOL PREPARATION FROM A CONDENSATION REACTION OF KETONE AND PHENOL

(71) Applicant: PTT Global Chemical Public Company Limited, Chatuchak, Bangkok (TH)

(72) Inventors: Papapida PORNSURIYASAK, Chatuchak, Bangkok (TH); Sarawut LUNVONGSA, Chatuchak, Bangkok (TH); Suchada TANG-AMORNSUKSAN, Chatuchak, Bangkok (TH)

(21) Appl. No.: 17/623,323

(22) PCT Filed: Jun. 17, 2020
(86) PCT No.: PCT/TH2020/000041

§ 371 Date: Dec. 28, 2021

(30) Foreign Application Priority Data
Jun. 28, 2019 (TH) 1901004088

(51) Int. Cl. C07C 37/20; C07C 37/82; B01J 31/10

(52) U.S. Cl. CPC C07C 37/20; C07C 37/82; B01J 31/10; B01J 2231/52

(57) ABSTRACT

The present invention relates to an isomerization process of product obtained from bisphenol preparation from a condensation reaction of ketone and phenol comprising contacting the product obtained from bisphenol preparation from a condensation reaction of ketone and phenol with the ion exchange resin in aqueous condition for the isomerization of the undesired product and separating the product having higher bisphenol content, wherein said ion exchange resin is aromatic polymer having the sulfonic acid group modified with at least one promoter selected from the compound shown in the structure (I):

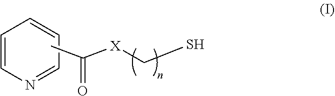

### TECHNICAL FIELD

The present invention relates to the field of chemistry, in particular, to the chemical mixture and chemical process in the preparation of ion exchange resin for the isomerization process of the undesired product obtained from bisphenol preparation.

### BACKGROUND ART

It is known that bisphenol is the substance obtained from the condensation reaction between phenol and ketone, especially 4,4′-isopropylidenediphenol (4,4′ BPA) which is the main desired bisphenol product to be used as the precursor in the preparation of polycarbonate. The production process of said bisphenol comprises the step of: (1) condensation reaction between acetone and phenol using acidic compound as the catalyst; (2) concentration of the condensation product by water removal; (3) crystallization of the 4,4′-isopropylidenediphenol product by cooling down the mixture obtained from step (2) and then separating the crystal of the 4,4′-isopropylidenediphenol from the mother liquor by filtration; (4) isomerization of the product obtained from the mother liquor after filtration in step (3) to be 4,4′-isopropylidenediphenol by contacting said liquid with the ion exchange resin comprising aromatic polymer having sulfonic acid group which may be modified with promoter; (5) return of liquid mixture from isomerization process in step (4) to step (2) in order to separate 4,4′-isopropylidenediphenol; (6) separation of phenol and bisphenol mixture in order to send back into the system for use and separation of tar generated from the side reaction in the process from the system; and (7) solidification and drying of the liquid 4,4′-isopropylidenediphenol product.

Generally, the product obtained from the production of bisphenol contains many compounds especially the 2,4′-isopropylidenediphenol which is the main product from the condensation reaction between acetone and phenol. It has been reported that the isomerization reaction to bisphenol product in the form of 4,4′-isopropylidenediphenol can occur by using acid as the catalyst and performing under the suitable condition. The acid used can be inorganic acid such as sulfuric acid and hydrochloric acid. Nevertheless, said acid catalyst still has some unwanted properties such as corrosion at the equipment, low percentage of converting obtained product to main product, and low selectivity of the production of desired product. At present, there have been many reports that the use of ion exchange resin having acid group as the catalyst or ion exchange resin immobilized with the mercaptan promoter, wherein the immobilization may be via ionic bond or chemical bond, can be used for the isomerization of the product obtained from bisphenol production.

The patent document no. U.S. Pat. No. 4,590,303 discloses the use of ion exchange resin immobilized with the mercaptan compound together with the acetone addition into the reactor during isomerization reaction. However, although the percentage of acetone conversion was increased, other products obtained would be largely formed too. Moreover, there is no report that the acetone conversion which is increased affects the increment of the amount of 4,4′-isopropylidenediphenol product.

The patent document no. U.S. Pat. No. 4,825,010 discloses the use of the ion exchange resin subjected to surface modification with mercaptoamine compound containing at least 2 branched chains of the alkyl mercaptan in the isomerization reaction. It was found capable to isomerize products such as 2,4′-isopropylidenediphenol and others without increase in the formation of compound from side reaction during isomerization such as 1,3,3-trimethyl-p-hydro-xyphenol-6-hydroxyindane and 4-methyl-2,4-bis-(4′-hydroxyphenyl)-pentene. However, this patent document does not indicate the increase in the amount of 4,4′-isopropylidenediphenol product.

The patent document no. U.S. Pat. No. 5,001,281 discloses the use of perfluoropolymer having sulfonic acid group in its structure as the isomerization catalyst for the production of bisphenol fluorene. It was found that the obtained products such as 2,4′-bisphenolfluorene and 9-fluorenone can be isomerized into 4,4′-bisphenolfluorene. However, there is no report that the use of said catalyst and process in the isomerization reaction can reduce the amount of compound from side reaction.

The patent document no. U.S. Pat. No. 4,822,923 discloses the use of ion exchange resin having sulfonic acid group as the catalyst with the use of alkylmercaptan promoter in the form of co-feed promoter in the isomerization reaction. It was found capable to isomerize the obtained products such as 2,4′-isopropylidenediphenol and others without increase in the formation of compound from side reaction during isomerization such as 1,3,3-trimethyl-p-hydro-xyphenol-6-hydroxyindane and 4-methyl-2,4-bis-(4′-hydroxyphenyl)-pentene. Nevertheless, this patent document does not indicate the increase in the amount of 4,4′-isopropylidenediphenol product and has the disadvantage in the industrial application because of the contamination of the promoter when being used. Therefore, the other further process is needed to separate the promoter from the obtained products.

The development of the production process of bisphenol still requires to reduce the amount of the obtained product, or to have the step that is effective to convert said obtained product to 4,4′-isopropylidenediphenol product and to reduce the amount of tar generated from the decomposition. This present invention aims to develop the isomerization process of product obtained from bisphenol preparation from a condensation reaction of ketone and phenol using ion exchange resin comprising aromatic polymer having the sulfonic acid group modified with promoter selected from the compound shown in the structure (I). The isomerization process of said product obtained increases the amount of bisphenol especially 4,4′-isopropylidenediphenol and reduces the amount of tar in the production process, which increases the ratio of product to precursor.

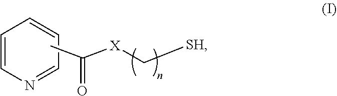

wherein,

X represents heteroatoms; and

n is an integer from 1 to 4.

### SUMMARY OF INVENTION

The present invention relates to an isomerization process of product obtained from bisphenol preparation from a condensation reaction of ketone and phenol comprising contacting the product obtained from bisphenol preparation from a condensation reaction of ketone and phenol with the ion exchange resin in aqueous condition for the isomerization of the undesired product and separating the product having higher bisphenol content, wherein said ion exchange resin is aromatic polymer having the sulfonic acid group modified with at least one promoter selected from the compound shown in the structure (I):

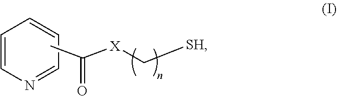

wherein,

X represents heteroatoms; and

n is an integer from 1 to 4.

### DESCRIPTION OF THE INVENTION

The present invention relates to the isomerization process of product obtained from bisphenol preparation from a condensation reaction of ketone and phenol using ion exchange resin comprising the aromatic polymer having the sulfonic acid group modified with the promoter.

Any aspect being described herein also means to include the application to other aspects of this invention unless stated otherwise.

### Definitions

Technical terms or scientific terms used herein have definitions as understood by an ordinary person skilled in the art unless stated otherwise.

Any tools, equipment, methods, or chemicals named herein mean tools, equipment, methods, or chemicals being operated or used commonly by those person skilled in the art unless stated otherwise that they are tools, equipment, methods, or chemicals specific only in this invention.

Use of singular noun or singular pronoun with “comprising” in claims or specification means “one” and also including “one or more”, “at least one”, and “one or more than one”.

All compositions and/or methods disclosed and claims in this application are intended to cover embodiments from any operation, performance, modification, or adjustment any factors without any experiment that significantly different from this invention, and obtain with object with utility and resulted as same as the present embodiment according to person ordinary skilled in the art although without specifically stated in claims. Therefore, substitutable or similar object to the present embodiment, including any minor modification or adjustment that can be apparent to person skilled in the art should be construed as remains in spirit, scope, and concept of invention as appeared in appended claims.

Throughout this application, term “about” means any number that appeared or expressed herein that could be varied or deviated from any error of equipment, method, or personal using said equipment or method, including variations or deviations occurred from changes in reaction conditions of uncontrollable factors such as humidity and temperature.

Heteroatom means to include the atom of the non-carbon element and said atom includes but not limited to tetrels group elements which are silicon, germanium, tin, and lead; pniktogens group elements which are nitrogen, phosphorus, arsenic, antimony, and bismuth; chalcogens group elements which are oxygen, sulfur, selenium, and tellurium; or halogens group elements which are fluorine, chlorine, bromine, and iodine.

Hereafter, invention embodiments are shown without any purpose to limit any scope of the invention.

The present invention relates to the isomerization process of product obtained from bisphenol preparation from a condensation reaction of ketone and phenol comprising contacting the product obtained from bisphenol preparation from a condensation reaction of ketone and phenol with the ion exchange resin in aqueous condition for the isomerization of the undesired product and separating the product having higher bisphenol content, wherein said ion exchange resin is aromatic polymer having the sulfonic acid group modified with at least one promoter selected from the compound shown in the structure (I):

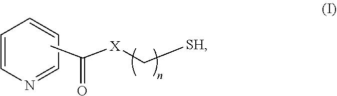

wherein,

X represents heteroatoms; and

n is an integer from 1 to 4.

Preferably, at least one promoter is the compound shown in the structure (II) or (III):

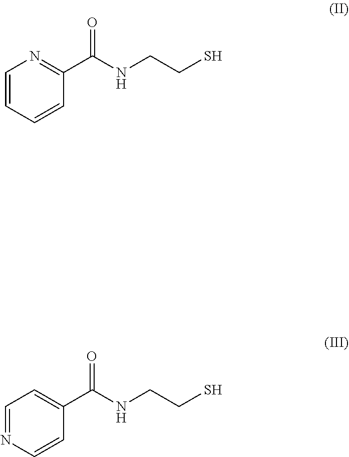 [SMILES: O=C(NCCS)c1ccccn1 ; O=C(NCCS)c1ccncc1]

More preferably, the promoter is the compound shown in the structure (II):

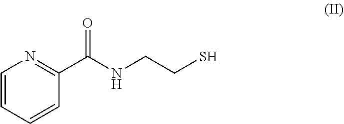 [SMILES: O=C(NCCS)c1ccccn1]

In one aspect, said promoter further comprises the aminoalkyl mercaptan which may be selected from cysteamine, 2-amino-1-propanethiol, and 3-mercaptopropylamine. Preferably, the aminoalkyl mercaptan is cysteamine.

Most preferably, the promoter is the mixture of the compound according to the structure (II) and cysteamine.

In one aspect of the invention, the promoter comprises the aminoalkyl mercaptan in the amount of 10 to 90% by mole of total promoter, preferably 30 to 70% by mole of total promoter.

In one aspect of the invention, the sulfonic acid group modified with promoter is 10 to 20% of total sulfonic acid group.

In one aspect, the sulfonic acid group is modified with promoter via ionic bond which may be formed via the nitrogen atom of said promoter and the sulfonic acid group of aromatic polymer.

Generally, said modification may be performed by mixing between the aromatic polymer having sulfonic acid group and the promoter, wherein water or aromatic hydroxy compound is used as the solvent.

In one aspect of the invention, the aromatic polymer having sulfonic acid group may be selected from polystyrene having sulfonic acid group or styrene-divinylbenzene copolymer having sulfonic acid group, preferably styrene-divinylbenzene copolymer having sulfonic acid group.

In one aspect, said styrene-divinylbenzene copolymer having sulfonic acid group may have divinylbenzene in the amount of about 1 to 15%, preferably in the amount of about 10 to 15%.

In one aspect, said aromatic polymer having sulfonic acid group has particle size in the range of about 500 to 1500 micron, preferably in the range of about 600 to 850 micron.

In one aspect of the invention, the weight ratio of ion exchange resin to product obtained from the bisphenol preparation is in the range of 1:1 to 1:5, preferably in the range of 1:1 to 1:3.

In one aspect of the invention, said isomerization reaction is performed at the temperature in the range of 40 to 120° C. and at atmospheric pressure, preferably at the temperature in the range of 60 to 80° C. and at atmospheric pressure.

In one aspect of the invention, said isomerization reaction is performed in aqueous condition having water content in the range of 0.01 to 3.5% by weight, preferably in the range of 0.05 to 0.5% by weight.

In one aspect, the product obtained from the bisphenol preparation before occurring isomerization reaction comprises the undesired products which are 4,4′-isopropylidenediphenol and 2,4′-isopropylidenediphenol.

In one aspect of the invention, the bisphenol preparation from a condensation reaction of ketone and phenol, wherein ketone may be selected from, but not limited to acetone, diethyl ketone, methyl ethyl ketone, ethylmethyl ketone, isobutylmethyl ketone, cyclohexanone, acetophenone, and 1,3-dichloroacetone; and phenol may be selected from, but no limited to unsubstituted phenol, alkylphenol, alkoxyphenol, naphthol, alkylnaphthol, and alkoxynaphthol.

In one aspect, the ion exchange resin used in isomerization reaction according to the invention may be used in the form of slurry for reaction in batch system or may be used in the form of fixed bed solid for reaction in continuous system.

In one aspect, the reaction time may be in the range of about 1 to 24 hours for reaction in batch system and reaction in continuous system. The liquid hourly space velocity (LHSV) may be in the range of about 0.015 to 10 hour⁻¹.

In one aspect of the invention, the separation of product having higher bisphenol content is the crystallization of the 4,4′-isopropylidenediphenol product in order to separate 4,4′-isopropylidenediphenol having high purity.

The following examples are for demonstrating the aspects of the invention only and not intended to be limitation of the scope of this invention in any way.

Preparation of the Promoter for Preparing Ion Exchange Resin According to the Invention

Compound as Shown in the Structure (II)

About 5 g of 2-pyridene carboxylic acid and about 6 g of cysteamine hydrochloride were dissolved in about 50 mL of dichloromethane solvent under an argon atmosphere. Then, the mixture solution of about 17 g of dicyclohexyl carbodiimide and about 1 g of 4-dimethylaminopyridene was added into about 20 mL of dichloromethane solvent. The said mixture was stirred at room temperature under an argon atmosphere for about 24 hours. The solid was filtered from the obtained mixture. Then, the filtrated liquid was evaporated under vacuum. After that, the filtrated liquid was crystallized in organic solvent or subjected to the purification using silica column. The obtained solid was separated and dried.

Preparation of the Ion Exchange Resin According to the Invention

The styrene-divinylbenzene copolymer type aromatic polymer having sulfonic acid group Amberlyst36 (Am36) with particle size in the range of about 600 to 850 micron was used in the preparation of the ion exchange resin according to the invention.

The promoter was dissolved in about 20 mL of deionized water and then added into about 20 g of styrene-divinylbenzene copolymer suspended in the deionized water. Then, said mixture was stirred at room temperature for about 1 hour. After stirring, the obtained mixture was packed in the glass column and washed with deionized water. The obtained solid was analyzed for the residual sulfonic acid group by titration method with sodium hydroxide solution in order to determine the sulfonic acid group (SO₃H) content before and after aromatic polymer modification. The percentage of sulfonic acid group modified with promoter could be calculated from the following equation.

$$Percentage of sulfonic acid group modified with promoter = \frac{mole of {SO}_{3} H before modification - mole of {SO}_{3} H after modification}{mole of {SO}_{3} H before modification} \times 100$$

Testing of the Isomerization Reaction Using Ion Exchange Resin

The ion exchange resin according to the invention was used as the catalyst for testing the isomerization efficiency of the product obtained from the bisphenol preparation by the following method.

Prior to use, the ion exchange resin was dehydrated by contacting about 10 g of the ion exchange resin with about 100 g of phenol at the temperature about 60 to 70° C. for about 15 minutes. This step was repeated for 3 times. Then, about 1.5 g of the obtained ion exchange resin was added into the round bottom flask. About 3 g of product obtained from the bisphenol preparation from a condensation reaction of ketone and phenol was contacted with said ion exchange resin in aqueous condition having desirable water content and then heated at desirable temperature. Then, the isomerization reaction of undesired product was operated until the end of desirable time.

The followings are the examples for testing of product compositions obtained from isomerization reaction using the ion exchange resin according to the invention, wherein methods and equipment used in testing are methods and equipment being used commonly and not intended to limit the scope of the invention.

The compositions and amounts of the products were analyzed by High Performance Liquid Chromatography (Shimadzu LC-20AD) using Reverse phase Phenomenex Gemini-NX 5μ C18 column and the solvent system between water and acetonitrile as the mobile phase with a flow rate of about 1 mL/min.

The % conversion of 2,4′-isopropylidenediphenol (2,4′ BPA), % weight gain of 4,4′-isopropylidenediphenol (4,4′ BPA) and % tar reduction were calculated from the following equations.

$$\% conversion of 2 , {4}^{′} - isopropylidenediphenol = \frac{signal of 2 , {4}^{′} - isopropylidenediphenol in ( feed - mixture after reaction )}{signal of 2 , {4}^{′} - isopropylidenediphenol in feed} \times 100$$

$$\% weight gain of 4 , {4}^{′} - isopropylidenediphenol = \frac{signal of 4 , {4}^{′} - isopropylidenediphenol in ( mixture after reaction - feed )}{signal of 4 , {4}^{′} - isopropylidenediphenol in feed} \times 100$$

$$\% tar reduction = \frac{tar content in feed - tar content in mixture after reaction}{tar content in feed} \times 100$$

TABLE 1
% conversion of 2,4′-isopropylidenediphenol and % weight gain of 4,4′-isopropylidenediphenol
after isomerization reaction using different ion exchange resins

| | | Water | | | % conversion of | % weight gain | |
|---|---|---|---|---|---|---|---|
| | | content | | | 2,4′- | of 4,4′- | |
| | | (% by | Temperature | Time | isopropylidene | isopropylidene | % tar |
| Sample | Promoter | weight) | (° C.) | (hr) | diphenol | diphenol | reduction |
| Comparative | — | 0.39 | 80 | 8 | 85.1 | 0 | 33.5 |
| sample 1 | | | | | | | |
| Comparative | — | 0.14 | 80 | 19 | 85.8 | 1.9 | 23.8 |
| sample 2 | | | | | | | |
| Comparative | — | 1.04 | 80 | 12 | 58 | 2.8 | 6.5 |
| sample 3 | | | | | | | |
| Comparative | — | 0.39 | 80 | 24 | 82.8 | 4.4 | 30.4 |
| sample 4 | | | | | | | |
| Comparative | cysteamine | 0.39 | 80 | 24 | 79.1 | 1.1 | 9.7 |
| sample 5 | | | | | | | |
| Comparative | — | 0.39 | 100 | 24 | 91.7 | 0.2 | 8.4 |
| sample 6 | | | | | | | |
| Sample | (II) + | 0.39 | 80 | 12 | 80.3 | 14.5 | 16.8 |
| according to | cysteamine | | | | | | |
| the invention | | | | | | | |
| 1 | | | | | | | |
| Sample | (II) | 0.39 | 80 | 12 | 67.4 | 1.7 | 8.5 |
| according to | | | | | | | |
| the invention | | | | | | | |
| 2 | | | | | | | |
| Sample | (II) + | 0.39 | 80 | 24 | 84.8 | 38.5 | 21 |
| according to | cysteamine | | | | | | |
| the invention | | | | | | | |
| 3 | | | | | | | |
| Sample | (II) + | 0.39 | 100 | 24 | 93.5 | 2.2 | 24.6 |
| according to | cysteamine | | | | | | |
| the invention | | | | | | | |
| 4 | | | | | | | |

From table 1, when comparing the comparative sample 1 to 5 to the sample according to the invention 1 to 4 which were subjected to test efficiency by isomerization reaction at temperature about 80° C., it was found that the ion exchange resin according to the invention gave high percent conversion of 2,4′-isopropylidenediphenol and percent weight gain of 4,4′-isopropylidenediphenol. The ion exchange resin according to the invention using the promoter shown in the structure (II) and cysteamine showed highest efficiency.

Moreover, when comparing the comparative sample 6 to the sample according to the invention 4 which were subjected to test efficiency by isomerization reaction at temperature about 100° C., it was found that the ion exchange resin according to the invention gave higher percent conversion of 2,4′-isopropylidenediphenol, percent weight gain of 4,4′-isopropylidenediphenol, and percent tar reduction.

From the results above, it can be said that the ion exchange resin according to the invention gives high efficiency for percent conversion of 2,4′-isopropylidenediphenol, percent weight gain of 4,4′-isopropylidenediphenol, and percent tar reduction as being stated in the objective of this invention.

### Best Mode or Preferred Embodiment of the Invention

Best mode or preferred embodiment of the invention is as provided in the description of the invention.

1. An isomerization process of a product obtained from a bisphenol preparation from a condensation reaction of a ketone and a phenol comprising contacting the product obtained from the bisphenol preparation from a condensation reaction of the ketone and the phenol with an ion exchange resin in aqueous condition for the isomerization of an undesired product comprising 2,4′-isopropylidenediphenol and separating a product having higher bisphenol content, wherein said ion exchange resin is an aromatic polymer having a sulfonic acid group modified with a promoter comprising:
a) at least one promoter selected from the compound shown in the structure (I): 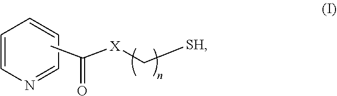
wherein,
X represents heteroatoms; and
n is an integer from 1 to 4, and
b) at least one promoter of an aminoalkyl mercaptan.

2. The process according to claim 1, wherein the promoter is the compound shown in the structure (II) or compound shown in the structure (III):

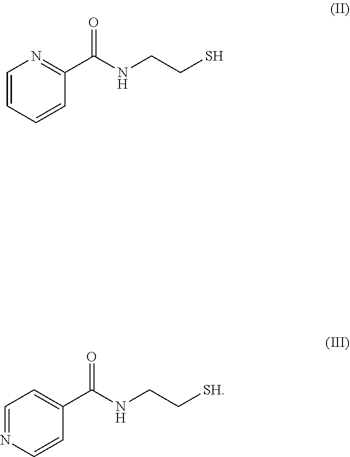 [SMILES: O=C(NCCS)c1ccccn1 ; O=C(NCCS)c1ccncc1]

3. The process according to claim 1, wherein the aminoalkyl mercaptan is cysteamine.

4. (canceled)

5. The process according to claim 13, wherein the promoter comprises the aminoalkyl mercaptan in the amount of 10 to 90% by mole of total promoter.

6. The process according to claim 5, wherein the promoter comprises the aminoalkyl mercaptan in the amount of 30 to 70% by mole of total promoter.

7. The process according to claim 1, wherein the sulfonic acid group modified with promoter is 10 to 20% of total sulfonic acid group.

8. The process according to claim 1, wherein the sulfonic acid group is modified with promoter via ionic bond.

9. The process according to claim 1, wherein the aromatic polymer having sulfonic acid group is selected from polystyrene having sulfonic acid group or styrene-divinylbenzene copolymer having sulfonic acid group.

10. The process according to claim 9, wherein the aromatic polymer having sulfonic acid group is the styrene-divinylbenzene copolymer having sulfonic acid group.

11. The process according to claim 10, wherein the styrene-divinylbenzene copolymer having sulfonic acid group has divinylbenzene in the amount of 1 to 15% by weight.

12. The process according to claim 11, wherein the styrene-divinylbenzene copolymer having sulfonic acid group has divinylbenzene in the amount of 10 to 15% by weight.

13. The process according to claim 1, wherein the aromatic polymer having sulfonic acid group has particle size in the range of 500 to 1500 micron.

14. The process according to claim 13, wherein the aromatic polymer having sulfonic acid group has particle size in the range of 600 to 850 micron.

15. The process according to claim 1, wherein the weight ratio of ion exchange resin to product obtained from the bisphenol preparation is in the range of 1:1 to 1:5.

16. The process according to claim 15, wherein the weight ratio of ion exchange resin to product obtained from the bisphenol preparation is in the range of 1:1 to 1:3.

17. The process according to claim 1, wherein said isomerization reaction is performed at the temperature in the range of 40 to 120° C. and at atmospheric pressure.

18. The process according to claim 17, wherein said isomerization reaction is performed at the temperature in the range of 60 to 80° C. and at atmospheric pressure.

19. The process according to claim 1, wherein said isomerization reaction is performed in aqueous condition having water content in the range of 0.01 to 3.5% by weight.

20. The process according to claim 19, wherein said isomerization reaction is performed in aqueous condition having water content in the range of 0.05 to 0.5% by weight.

21. The process according to claim 1, wherein the product obtained from the bisphenol preparation further comprises 4,4′-isopropylidenediphenol.
